(19) United States
(12) Patent Application Publication
(10) Pub. No.: US 2011/0263574 A1
(43) Pub. Date: Oct. 27, 2011

(54) PIRENZEPINE AS OTOPROTECTIVE AGENT

(75) Inventor: Andre Schrattenholz, Mainz (DE)

(73) Assignee: PROTEOSYS AG, Mainz (DE)

(21) Appl. No.: 13/144,342

(22) PCT Filed: Jan. 13, 2010
(86) PCT No.: PCT/EP2010/050348

§ 371 Date: Jul. 13, 2011

Related U.S. Application Data
(60) Provisional application No. 61/144,204, filed on Jan. 13, 2009.

(51) Int. Cl. A61K 31/5513; C07D 471/04; A61P 27/16

(52) U.S. Cl. 514/220; 540/495

(57) ABSTRACT

The present invention generally relates to the otoprotective activity of condensed diazepinones, e.g. condensed benzodiazepines such as pirenzepine or compounds which are metabolized to condensed benzodiazepinones such as olanzapine. These compounds are suitable as medicaments for the prevention and/or treatment of otic diseases, e.g. diseases associated with loss of hearing.

The present invention generally relates to the otoprotective activity of condensed diazepinones, e.g. condensed benzodiazepinones such as pirenzepine or compounds which are metabolized to condensed benzodiazepinones such as olanzapine. These compounds are suitable as medicaments for the prevention and/or treatment of otic diseases, e.g. diseases associated with loss of hearing.

Pirenzepine (5,11-dihydro-11[(4-methyl-1-piperazinyl)-acetylJ-6H-pyrido-[2,3-b]-[1,4] benzodiazepine-6-one), is a topical antiulcerative M1 muscarinic antagonist, that inhibits gastric secretion at lower doses than are required to affect gastrointestinal motility, salivary, central nervous system, cardiovascular, ocular, and urinary function. It promotes the healing of duodenal ulcers and due to its cytoprotective action is beneficial in the prevention of duodenal ulcer recurrence. It also potentiates the effect of other antiulcer agents such as cimetidine and ranitidine. It is generally well tolerated by patients. The M1 muscarinic effect of pirenzepine is thought to be an explanation for this and a variety of additional effects in other indications, listed below.

WO 2006/008118 and WO 2006/008119 describe that pirenzepine and related compounds are inhibitors of PARP and SIR2. The use of these compounds as cytoprotective, particularly neuroprotective agents, is disclosed. The contents of these documents is herein incorporated by reference.

The administration of ototoxic agents or noise trauma may mediate apoptosis and/or necrosis of sensoric cells due to oxidative stress (Henderson et al., Ear Hear. 27 (2006), 1-19). In early stages of apoptosis a massive activation of PARP-1 was detected (Yu et al., Science 297 (2002), 259-263). Further, it was found that PARP-1 activation causes a translocation of AIF (Apoptosis Inducing Factor) from the mitochondriae to the nucleus and an AIF-mediated PARP-1 dependent caspase-independent apoptosis (Yu et al., (2002), supra). PARP-1 hyperactivity is also associated with necrotic cell death (Virag and Szabo, Pharmacol Rev. 54 (2002), 375-429). Further it could be shown that the PARP-1 inhibitor 3-aminobenzamide alleviates cochleal dysfunctions induced by transient ischemia or acoustic trauma (Tabuchi et al., Ann. Otol. Rhinol. Laryngol. 110 (2001), 118-121; Tabuchi et al., J. Exp. Med. 200 (2003), 1995-2002).

According to the present invention it was found that pirenzepine and related compounds show significant otoprotective activity against administration of otoxitic drugs.

Thus, a first aspect of the present invention relates to the use of a compound of formula I

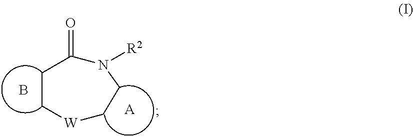

wherein A and B are five- or six-membered rings optionally containing at least one heteroatom selected from N, S and O, wherein the rings are optionally mono- or polysubstituted with halo, e.g. F, Cl, Br, or I, C₁-C₄-(halo)-alkyl, C₁-C₄-(halo)-alkoxy, amino, C₁-C₄-alkyl-amino, or di(C₁-C₄-alkyl)amino,

### W is S, O, NR¹ or CHR¹

R1 is hydrogen, Y or COY,
R2 is hydrogen or C₁-C₄-(halo)-alkyl, and
Y is C₁-C₆ (halo)alkyl, or C₃-C₈ cyclo-(halo)-alkyl, wherein the alkyl or cycloalkyl group is optionally substituted with a five- or six-membered ring optionally containing at least one heteroatom selected from N, S and O, and wherein the ring is optionally mono- or poly-substituted with halo, C₁-C₄-(halo)alkyl, C₁-C₄(halo)alkoxy, amino, C₁-C₄-alkyl amino, di(C₁-C₄-alkyl)amino or Z,
wherein Z is a C₁-C₆ (halo) alkyl group ω-substituted with a group N(R4)₂, wherein each R4 is independently hydrogen, C₁-C₈ alkyl, or CO—C₁-C₈-alkyl or wherein both R4 together form a five- or six-membered ring optionally containing at least one further heteroatom selected from N, S and O, wherein the ring is optionally mono- or polysubstituted with halo, C₁-C₄(halo)-alkyl and C₁-C₄(halo) alkoxy,
or of a salt or derivative thereof for the manufacture of an otoprotective medicament.

The term “(halo)alkyl” according to the present invention relates to an alkyl group which optionally contains at least one halo, e.g. F, Cl, Br or I substituent up to perhalogenation.

The term “salt” preferably refers to pharmaceutically acceptable salts of compounds of Formula I with suitable cations and/or anions. Examples of suitable cations are alkaline metal cations such as Li⁺, Na⁺ and K⁺, alkaline earth metal cations such as Mg⁺ and Ca⁺ as well as suitable organic cations, e.g. ammoniums or substituted ammonium cations. Examples of pharmaceutically acceptable anions are inorganic anions such as chloride, sulfate, hydrogen sulfate, phosphate or organic cations such as acetate, citrate, tartrate, etc.

Derivatives of compounds of Formula I are any molecules which are converted under physiological conditions to a compound of Formula I, e.g. esters, amides etc. of compounds of Formula I or molecules which are products of metabolization reactions of a compound of Formula I.

Preferably, the compounds of Formula I are used for the prevention or treatment of otic PARP-1 associated disorders, i.e. otic disorders which are caused by and/or accompanied by excitotoxicity and/or apoptosis, in particular mitochondrial apoptosis and/or calcium-related cell stress. For example, these disorders are selected from dysfunctions of middle or inner ear, e.g. cochleal disorders associated with partial or complete loss of hearing, particularly at higher frequency. Preferably, the invention refers to loss of hearing caused by aging, by noise trauma, e.g. by acute or chronic noise trauma, and/or by administration of ototoxic compounds, e.g. administration of chemotherapeutic agents, particularly platinum compounds such as cis-platinum or carboplatinum in cancer therapy or administration of antibiotics, such as aminoglycosides.

It was found that compounds of formula I prevent an irreversible loss of auditory sensory cells, e.g. outer or inner hair cells, which may be caused by and/or accompanied by aging, noise or toxic compounds.

For therapeutic applications, the compounds of Formula I may be used alone or together with other medicaments, e.g. together with other otoprotective medicaments such as other PARP-1 inhibitors and/or anti-excitatory medicaments such as memantine.

Particularly, the compounds of formula I may be administered to a subject who is under treatment with medicaments having ototoxic side effects, e.g. platinum compounds or aminoglycosides, in order to reduce and/or abolish the ototoxic side effects of such compounds.

Surprisingly, it was found that administration of the compounds of formula I does not negatively affect the cytotoxic anti-tumor activity of chemotherapeutic agents, e.g. cis-platinum.

In the compounds of Formula I, the cyclic groups A and B are preferably selected from

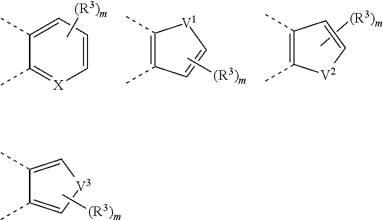

wherein X is N or CR3,
V1, V2 or V3 are selected from —O—, —S—, and NR6,
R3 is in each case independently halo, C₁-C₄-(halo)-alkyl, C₁-C₄-(halo)-alkyl, C₁-C₄-(halo)alkoxy, amino, C₁-C₄-alkyl-amino, or di(C₁-C₄-alkyl)amino,
m is an integer of 0-2, and
R6 is hydrogen or C₁-C₄-(halo)alkyl.

More preferably, the cyclic group A is selected from

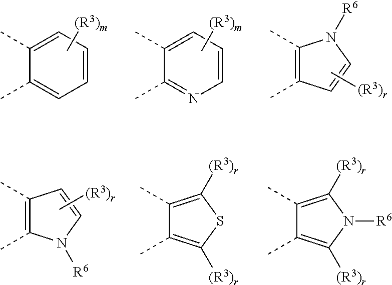

wherein R3 is defined as above,
m is an integer of 0-2,
r is an integer of 0-1 and
R6 is hydrogen or methyl.
More preferably, the cyclic group B is selected from

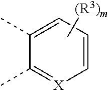

wherein X, R3 and m are as defined above

In one embodiment, R1 is Y. In this case Y is preferably C₃-C₈ cyclo(halo)-alkyl, e.g. cyclopropyl, cyclobutyl or cyclopentyl.

In a further embodiment, R1 is COY and Y is selected from

—(CHR7)q-R8

wherein R7 is hydrogen, halo or C₁-C₄-(halo)alkyl,
q is an integer of 1-4, and preferably 1 and
R8 is a five- or six-membered ring optionally containing at least one heteroatom, wherein the ring is optionally mono- or polysubstituted with C₁-C₄(halo)alkyl or a ω-amino-substituted alkyl group Z as defined above.

In this embodiment, R8 is preferably selected from

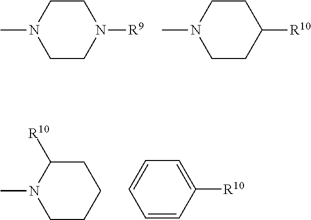

wherein R9 is hydrogen or C₁-C₄(halo)alkyl and R10 is a ω-amino-substituted alkyl group Z as defined above.

R9 is preferably a methyl group. The ω-amino-substituted alkyl group Z is preferably a C₁-C₄ (halo)alkyl group having a terminal amino group which is substituted with at least one C₁-C₆ alkyl group, e.g. a diethylamino, or diisobutylamino group, or with a CO(C₁-C₆) alkyl group and with hydrogen or a C₁-C₂ alkyl group.

Specific examples of compounds of Formula I are pirenzepine and related compounds as disclosed in FR 1,505,795, U.S. Pat. Nos. 3,406,168, 3,660,380, 4,021,557, 4,210,648, 4,213,984, 4,213,985, 4,277,399, 4,308,206, 4,317,823, 4,335,250, 4,424,222, 4,424,226, 4,724,236, 4,863,920, 5,324,832, 5,620,978, 6,316,423, otenzepad and related compounds as disclosed in U.S. Pat. Nos. 3,406,168, 5,324,832 and 5,712,269, AQ-RA741 and related compounds as disclosed in U.S. Pat. Nos. 5,716,952, 5,576,436 and 5,324,832, viramune and related compounds as disclosed in EP-A-0429987, and U.S. Pat. Nos. 5,366,972, 5,705,499, BIBN 99 and related compounds as disclosed in U.S. Pat. Nos. 6,022,683 and 5,935,781, DIBD, telenzepine and related compounds as disclosed in EP-A-0035519, and U.S. Pat. No. 4,381,301 and salts or derivatives thereof. The above documents are herein incorporated by reference.

Further preferred compounds are 7-azabicyclo-[2.2.1]-heptane and heptene compounds such as a tiotropium bromide as disclosed in U.S. Pat. Nos. 5,817,679, 6,060,473, 6,077,846, 6,117,889, 6,255,490, 6,403,584, 6,410,583, 6,537,524, 6,579,889, 6,608,055, 6,627,644, 6,635,658, 6,693,202, 6,699,866 and 6,756,392, heterocyclic compounds, e.g. pyrrolidinones, tetrahydropyridines, isoxazocarboxamides, thienopyrane carboxamides, or benzopyranes, such as alvameline tartrate and related compounds disclosed in U.S. Pat. Nos. 6,306,861, 6,365,592, 6,403,594, 6,486,163, 6,528,529, 6,680,319, 6,716,857 and 6,759,419, metocloproamide and related compounds as disclosed in U.S. Pat. No. 3,177,252 and QNB and related compounds as disclosed in U.S. Pat. No. 2,648,667 and salts and derivatives thereof. The above documents are herein incorporated by reference.

Further, the invention encompasses compounds which are metabolized to give diaryl diazepinones according to Formula I such as clozepine and olenzepine.

The compounds as indicated above are preferably administered to a subject in need thereof, e.g. a human subject, as a pharmaceutical composition, which may contain pharmaceutically acceptable carriers, diluents and/or adjuvants. The pharmaceutical composition may be administered in the form of a tablet, capsule, solution suspension, etc. The medicament may be administered according to any known means, wherein oral and intravenous administration is particularly preferred. Alternatively, the medicament may be directly administered to the ear.

The present application has applications in human and veterinary medicine, particularly in human medicine.

Furthermore, the present invention shall be explained by the following Figures and Examples.

### FIGURE LEGENDS

FIG. 1 shows Otoprotection by pirenzepine (PSY 310).

In cultivated cochlea, a loss of sensory or hair cells was induced by administering cis-platin (5 μM).

(A): Inner hair cells (IHC) and (B): Outer hair cells (OHC).
Left: Comparison of hair cell protection (preserved fraction) in wild-type (+/+) heterozygous (+/−) and homozygous (−/−) PARP-1 knock out mice.
Right: Addition of pirenzepine caused dosis dependent protection of sensory cells. The preserved cell fraction is significantly increased compared to controls.

FIG. 2 shows Otoprotection by LS 75 (PSY 3101).

In cultivated cochlea, a loss of sensory or hair cells was induced by administering cis-platin (5 μM).

(A): Inner hair cells (IHC) and (B): Outer hair cells (OHC).
Left: Comparison of hair cell protection (preserved fraction) in wild-type (+/+) heterozygous (+/−) and homozygous (−/−) PARP-1 knock out mice.
Right: Addition of LS 75 caused dosis dependent protection of sensory cells. The preserved cell fraction is significantly increased compared to controls.

FIG. 3 shows the survival rate of cis-platin (1.4 μM) treated cancer cell lines (germ cell tumors 2101 Ep and NT2) without (black bars) or with simultaneous administration (grey bars) of 10 μM PSY 301 (pirenzepine) or PSY 3103 (LS 75) compared to control (DMSO: 0.1%).

### EXAMPLES

### Example 1

### Otic Protectivity of Compounds PSY 310 (Pirenzepine) and PSY 3103 (LS 75)

### 1. Materials and Methods

Intact cochlea of post-natal mice were cultivated up to 7 days (Unsworth and Lelkes, Nat. Med. 4 (1998), 901-907) in simulated microgravity.

The otic protectivity of the test compounds PSY 310 and PSY 3101 in the presence of ototoxic agents was tested. Neomycin (an aminoglycoside antibiotic) and cis-platinum (a chemotherapeutic agent) were added in three different concentrations to the cultivate organ over a time period of 48 hours.

The test compounds were added in six different amounts of 0.1 to 100 μM respectively.

### 2. Results

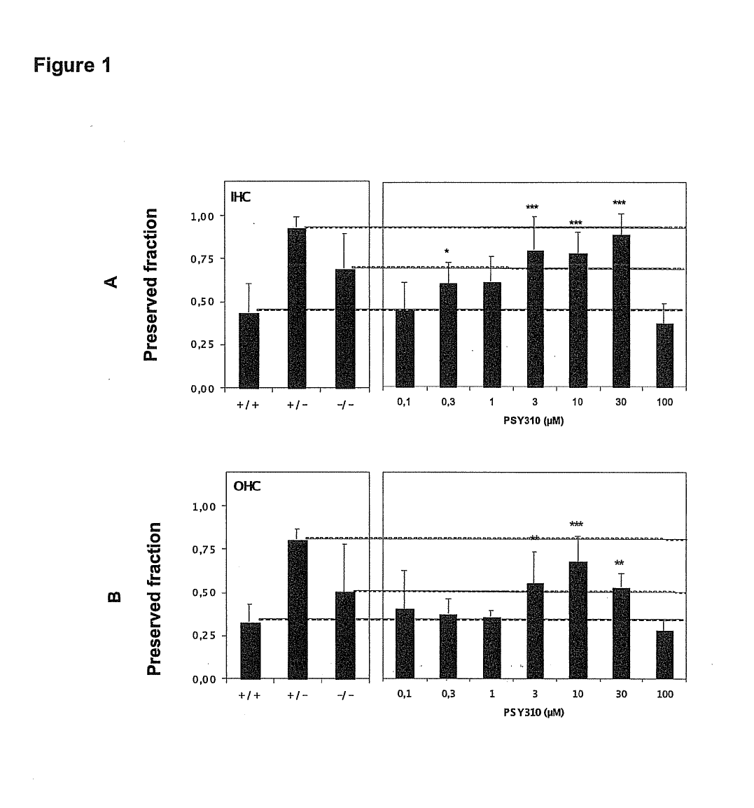

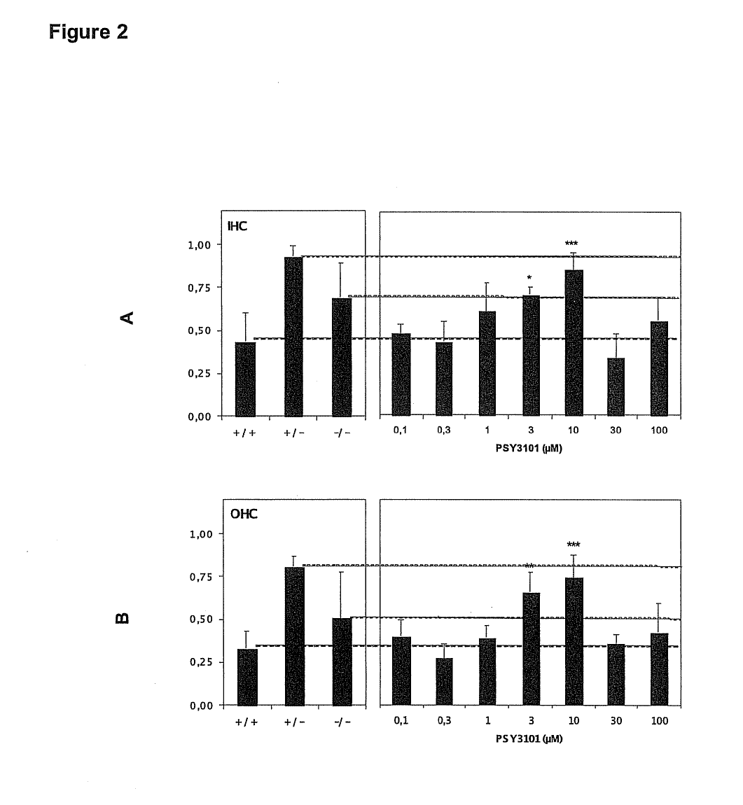

The results are shown in FIGS. 1 and 2. Administration of pirenzepine and LS 75 resulted in a dose-dependent increase of the preserved fraction of inner and outer hair cells from the ototoxic effect of cis-platinum.

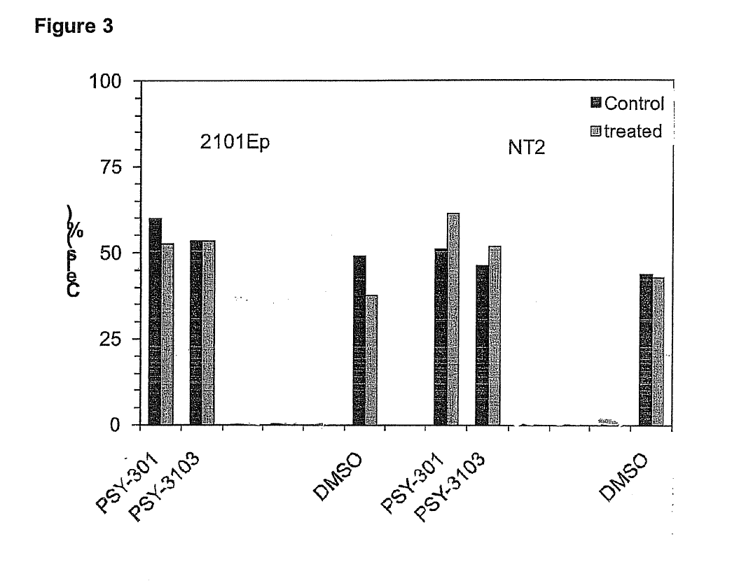

FIG. 3 shows that administration of pirenzepine and LS 75 does not reduce the (desired) cytotoxic effect of cis-platinum on germ cell tumor cell lines Ep 2101 and NT2.

1. Use of a compound of formula I 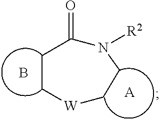
wherein A and B are a five- or six-membered ring optionally containing at least one heteroatom selected from N, S and O, wherein the ring is optionally mono- or polysubstituted with halo, C₁-C₄-(halo)-alkyl, C₁-C₄-(halo)-alkoxy, amino, C₁-C₄-alkyl-amino, or di(C₁-C₄-alkyl)amino,
W is S, O, NR₁ or CHR₁
R1 is hydrogen, Y or COY,
R2 is hydrogen or C₁-C₄-(halo)-alkyl, and
Y is C₁-C₆ (halo)alkyl, or C₃-C₈ cyclo-(halo)-alkyl, wherein the alkyl or cycloalkyl group is optionally substituted with a five- or six-membered ring optionally containing at least one heteroatom selected from N, S and O, wherein the ring is optionally mono- or poly-substituted with halo, C₁-C₄-(halo)alkyl, Cᵣ C₄(halo)alkoxy, amino, C₁-C₄-alkyl amino, di(C₁-C₄-alkyl)amino or Z,
wherein Z is a C₁-C₆ (halo) alkyl group ω-substituted with a group N(R4)₂, wherein each R4 is independently hydrogen, C₁-C₈ alkyl, or CO—C₁-C₈-alkyl or wherein both R4 together form a five- or six-membered ring optionally containing at least one further heteroatom selected from N, S and O, wherein the ring is optionally mono- or polysubstituted with halo, C₁-C₄(halo)-alkyl and C₁-C₄(halo) alkoxy,
or of a salt or derivative thereof for the manufacture of an otoprotective medicament.

2. The use of claim 1 for the manufacture of a medicament for the prevention or treatment of otic PARP-1-associated disorders.

3. The use of claim 1 for the manufacture of a medicament for the prevention or treatment of cochleal disorders associated with partial or complete loss of hearing particularly at higher frequency.

4. The use of claim 1 for the manufacture of a medicament for the prevention or treatment of loss of hearing caused by aging, by noise trauma and/or by administration of ototoxic compounds.

5. The use of claim 4 for the manufacture of a medicament for the prevention or treatment of loss of hearing caused by administration of chemotherapeutic agents, particularly platinum compounds such as cis-platin, or carboplatinum, or antibiotics, particularly aminoglycoside antibiotics.

6. The use of claim 1 for administration to a subject who is under treatment of medicaments having ototoxic side effects.

7. The use of claim 1 wherein the cyclic groups A and B are selected from 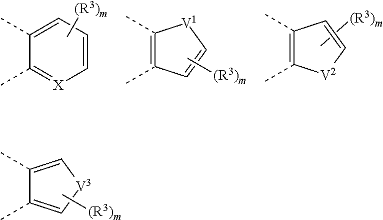
wherein X is N or CR3,
V1, V2 or V3 are selected from —O—, —S—, and NR6,
R3 is halo, C₁-C₄-(halo)-alkyl, C₁-C₄-(halo)-alkoxy, amino, C₁-C₄-alkyl-amino, or di(C₁-C₄-alkyl)amino,
m is an integer of 0-2, and
R6 is hydrogen or C₁-C₄-(halo)alkyl.

8. The use of claim 7, wherein the cyclic groups A and B are selected from 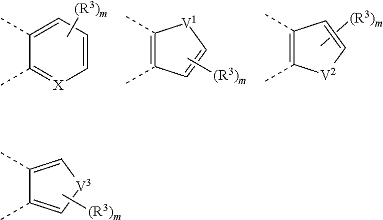
wherein R3 is halo, C₁-C₄-(halo)-alkyl, C₁-C₄-(halo)-alkoxy, amino, C₁-C₄-alkyl-amino, or di(C₁-C₄-alkyl) amino,
m is an integer of 0-2,
r is an integer of 0-1 and
R6 is hydrogen or methyl.

9. The use of claim 1 wherein R1 is Y and Y is C₃-C₈-cyclo(halo)alkyl.

10. The use of claim 1 wherein R1 is COY and Y is selected from
—(CHR7)q-R8
wherein R7 is hydrogen, halo or C₁-C₄-(halo)alkyl,
q is an integer of 1-4, and preferably 1 and
R8 is a five- or six-membered ring optionally containing at least one heteroatom, wherein the ring is optionally mono- or polysubstituted with C₁-C₄(halo)alkyl or a ω-amino-substituted alkyl group Z.

11. The use of claim 10 wherein R8 is selected from 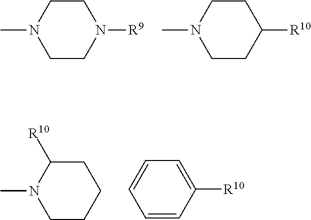
wherein R9 is hydrogen or C₁-C₄(halo)alkyl and R10 is a ω-amino-substituted alkyl group Z, wherein Z is a C₁-C₆ (halo) alkyl group ω-substituted with amu N(R4)₂, wherein each R4 is independently hydrogen, C₁-C₈ alkyl, or CO—C₁-C₈-alkyl or wherein both R4 together form a five- or six-membered ring optionally containing at least one further heteroatom selected from N, S and O, wherein the ring is optionally mono- or polysubstituted with halo, C₁-C₄(halo)-alkyl and C₁-C₄(halo) alkoxy.

12. The use of claim 1 wherein the compound of Formula I is selected from pirenzepine LS-75, otenzepad, AQ-RA741, viramune, BIBN 99, DIBD, telenzepine and salts or derivatives thereof.

13. The use of claim 1 for use in human medicine.

14. A method of treating an otic PARP-1-associated disorder in a patient in need of such treatment, comprising administering to said patient an effective amount of a compound of formula I of claim 1.
